(19) United States
(12) Patent Application Publication
(10) Pub. No.: US 2002/0173474 A1
(43) Pub. Date: Nov. 21, 2002

(54) Methods & materials involving dimerization-mediated regulation of biological events

(75) Inventors: Stuart L. Schreiber, Boston, MA (US); Gerald R. Crabtree, Woodside, CA (US)

Correspondence Address: David L. Berstein, ARIAD Pharmaceuticals, Inc., 26 Landsdowne Street, Cambridge, MA, 02139-4234, US

(73) Assignee: President and Fellows of Harvard College

(21) Appl. No.: 09/834,424

(22) Filed: Apr. 13, 2001

Related U.S. Application Data
(62) Division of application No. 09/430,508, filed on Oct. 29, 1999.
Application No. 09/430,508 is a continuation-in-part of application No. 09/087,716, filed on May 29, 1998, now Pat. No. 6,011,018.
Application No. 09/087,716 is a continuation-in-part of application No. 08/388,653, filed on Feb. 14, 1995, now Pat. No. 5,869,337.
Application No. 08/388,653 is a continuation-in-part of application No. 08/196,043, filed on Feb. 14, 1994, now abandoned.
Application No. 08/196,043 is a continuation-in-part of application No. 08/179,748, filed on Jan. 7, 1994, now abandoned.
Application No. 08/179,748 is a continuation-in-part of application No. 08/092,977, filed on Jul. 16, 1993, now abandoned.
Application No. 08/092,977 is a continuation-in-part of application No. 08/017,931, filed on Feb. 12, 1993, now abandoned.
(63) Continuation-in-part of application No. 08/973,337, filed on Nov. 28, 1997, now abandoned.
Application No. 08/973,337 is a continuation-in-part of application No. 08/480,286, filed on Jun. 7, 1995, now abandoned.
Application No. 08/480,286 is a continuation-in-part of application No. 08/400,800, filed on Mar. 7, 1995, now abandoned.
Application No. 08/400,800 is a continuation-in-part of application No. 08/332,995, filed on Nov. 1, 1994, now abandoned.

(30) Foreign Application Priority Data
Nov. 1, 1995 (US) PCTUS9514177

(51) Int. Cl. A61K 48/00

(52) U.S. Cl. 514/44

(57) ABSTRACT

This invention concerns materials, methods and applications relating to the multimerizing of protein mediators of biological events using synthetic, preferably non-peptidic, dimerizing agents.

### RELATED APPLICATIONS

This application is a divisional application of U.S. Ser. No. 09/430,508 (filed Oct. 29, 1999) which is a continuation in part of U.S. Ser. No. 09/087,716 (filed May 29, 1998, now U.S. Pat. No. 6,011,018), which in turn is a continuation in part of U.S. Ser. No. 08/388,653 (filed Feb. 14, 1995, now U.S. Pat. No. 5,869,337), which in turn is a continuation in part of U.S. Ser. No. 08/196,043 (filed Feb. 11, 1994), which in turn is a continuation in part of U.S. Ser. No. 08/179,748 (filed Jan. 7, 1994), which in turn is a continuation in part of U.S. Ser. No. 08/092,977 (filed (Jul. 16, 1993), which in turn is a continuation in part of U.S. Ser. No. 08/017,931 (filed Feb. 12, 1993).

### STATEMENT OF RIGHTS

This invention involves work supported by the US Government, which therefore has certain rights therein.

### TECHNICAL FIELD

This invention concerns materials, methods and applications thereof relating to the multimerizing of protein mediators of biological events, using dimerizing agents which are nonpeptidic, less than 5 kD in molecular weight and/or membrane permeant.

### BACKGROUND

### Receptor Dimerization—a Big Molecule Job

Biological specificity usually results from highly specific interactions among proteins. This principle is exemplified by signal transduction, the process by which extracellular molecules influence intracellular events. Many signaling pathways are triggered by the binding of extracellular ligands to cell surface receptors. Examples include the binding of a variety of polypeptides (e.g., hormones, growth factors and cytokines) to their receptors. Such ligand binding promotes dimerization or clustering of receptor molecules. In many cases receptor dimerization leads to transphosphorylation and the recruitment of proteins that continue the signaling cascade. Receptor activation through homodimerization was confirmed by the experimental activation of cell surface receptors using antibodies that cross linked two receptor molecules. Subsequently, many receptors were found to become activated upon dimerization or oligomerization. The extracellular and transmembrane regions of many receptors are believed to function by bringing the cytoplasmic domains of the receptor molecules into close proximity with one another through a ligand-dependent dimerization or oligomerization, while the cytoplasmic domains of the receptor convey specific signals to internal compartments of the cell.

A considerable amount of research has now been directed to the identification and characterization of protein-protein interactions involved in mediating a variety of biological events. Many research groups in academic and industrial laboratories have focused their efforts on inhibiting certain protein-protein interactions which are believed to mediate disease processes.

In a departure from those efforts, and from research exploring dimerization induced by protein hormones or antibodies, our work has led to a generally applicable toolkit of materials and methods for using small molecules to promote homodimerization, heterodimerization and oligomerization of proteins in living cells to regulate biological events.

We demonstrated the feasibility and power of biological regulation based on small-molecule-mediated multimerization using a model system employing chimeric receptor proteins. That work led to the development of technology of great potential utility in biological research and in gene and cell therapies. In that system, chimeric proteins containing a specific receptor domain are expressed in cells. Treatment of the cells with a small molecule, multivalent ligand which binds to the receptor domain leads to dimerization or oligomerization of the chimeric protein molecules. By analogy to other chimeric receptors (see e.g. Weiss, Cell (1993) 73, 209), the chimeric proteins are designed such that oligomerization triggers the desired subsequent events, e.g. the propagation of an intracellular signal. Some aspects of that work are disclosed below to illustrate certain embodiments of the subject invention. For additional background information and guidance, see, e.g., Spencer et al, Nov. 12, 1993, Science 262:1019-102; U.S. Pat. Nos. 5,830,462 and 5,871,753; and numerous subsequent scientific papers and patent documents.

That body of work established the utility of small molecule-dependent oligomerization as a regulatory mechanism, demonstrating among other points, the applicability of the system to a variety of signaling pathways, the utility of the approach in mammals, and the feasibility of identifying and deploying ligands for proteins of interest.

The subject invention draws upon the same tool kit and applies many of the same principles to endogenous proteins and signaling pathways.

### SUMMARY OF THE INVENTION

### A New Drug Discovery Paradigm

Dimerization and oligomerization of proteins are general biological control mechanisms that contribute to the activation of cell surface receptors, transcription factors, vesicle fusion proteins and other classes of intra- and extracellular proteins. We have developed a general procedure for the regulated (inducible) dimerization or oligomerization of intracellular proteins. This is accomplished using ligands, preferably “small molecule” ligands, which can bind to and cross-link two or more protein molecules endogenous to the cells, i.e., proteins native to a cell or invading organism thereof. Such multivalent ligands which, as described herein, promote the association of endogenous proteins in cells to effect a biological event have been referred to as “chemical inducers of dimerization” (CIDs), or simply “dimerizers”.

In principle, any two target proteins can be induced to associate by treating the cells or organisms that harbor them with an appropriate dimerizer, preferably a cell permeant, synthetic dimerizer. To illustrate the practice of this invention, we have induced: (1) the intracellular aggregation of the cytoplasmic tail of the zeta chain of the T cell receptor (TCR)-CD3 complex thereby leading to signaling and transcription of a reporter gene, (2) the homodimerization of the cytoplasmic tail of the Fas receptor thereby leading to cell-specific apoptosis (programmed cell death) and (3) the heterodimerization of a DNA-binding domain (Gal4) and a transcription-activation domain (VP16) thereby leading to direct transcription of a reporter gene. Those oligomerization-based studies were conducted using fusion proteins which were dimerized or oligomerized in a model for the dimerization of endogenous cellular proteins. Other illustrations include the homodimerization of receptor proteins for EPO, G-CSF, TPO, GH, IL-2, IFN-alpha, IFN-beta or insulin and the heterodimerization of HIV protease with topoisomerase I, ZAP-70 with topoisomerase I, and a viral protein with a cellular proteosomal protein.

Regulated intracellular protein association, as described herein, offers new capabilities in biological research and medicine and represents a new paradigm in drug discovery and pharmaceutical therapies.

Accordingly, one object of this invention is a method for activating a signal transduction pathway which is mediated by a complex of endogenous proteins, e.g. a dimerized or oligomerized cell surface receptor; a dimerized or oligomerized receptor for a polypeptide growth factor, cytokine or hormone; or other endogenous protein complex. The method involves contacting a cell with a dimerizer that promotes the formation of protein complexes which activate the pathway of interest.

Receptors of particular interest include receptors for EPO, G-CSF, TPO, GH, IL-2, interferon-alpha, interferon-beta, insulin and neurotropic factors. In such cases, this invention provides a method for activating a signal transduction pathway of such receptors by contacting a cell with a multivalent dimerizer that binds to more than one molecule of the receptor.

Dimerizers of this invention have one or more of the following characteristics: they are nonpeptidic, less than 5 kD in molecular weight and membrane permeant. The dimerizer will be capable of binding to at least two molecules of the receptor protein or other endogenous protein, and in many cases comprises at least two receptor-binding moieties covalently linked together. The dimerizer may bind to a cytoplasmic or extracellular portion of the receptor. In certain embodiments, the dimerizer binds to the receptor with a Kd<10⁻⁶M. Preferably the dimerizer has a molecular weight less than 5 kD. Preferably the dimerizer is nonpeptidic.

In in vitro applications where the cells are present in a culture medium, contacting the cells with the dimerizer is effected by adding the dimerizer to the culture medium.

In applications where the cells are present in a host organism, e.g. a mammal, the contacting is effected by administering the dimerizer to the host organism. In such cases, it will generally be preferable to use a composition comprising a dimerizer in admixture with a pharmaceutically acceptable carrier and optionally with one or more pharmaceutically acceptable excipients.

Another object of this invention is a method for preparing a dimerizer as described herein. One such method involves covalently linking a first compound capable of binding to one of the endogenous protein mediators with a second compound capable of binding to the other protein mediator, to form a dimerizing agent capable of binding to both mediator molecules. Note that in cases involving homodimerization and homo-oligomerization, the first and second compounds may be the same. Preferably the first and second compounds are non-peptidic.

This method may be applied to protein mediators including a cell surface receptor for a cytokine, growth factor or other hormone. Applying this method to receptors for EPO, G-CSF, TPO, GH, IL-2, interferon-alpha, interferon-beta, insulin or a neurotropic factor are of particular interest.

The method may also be applied to cases where the biological event to be triggered is mediated by the association of molecules of two different protein mediators. In such cases, the first and second compounds are different from one another.

Dimerizers so prepared may be formulated into pharmaceutical compositions and/or used as described above.

Biological events which can be triggered by a dimerizer of this invention include, among others, cellular growth, proliferation or differentiation as well as gene transcription, translocation of a selected protein to a predetermined cellular compartment, or destruction of a selected protein.

### BRIEF DESCRIPTION OF THE DRAWINGS

FIG. 1 depicts the identification of receptor binding compounds and their use in the design of dimerizers.

FIG. 2 depicts the use of the dimerizer, FK1012, to trigger signaling in cells expressing an FKBP-CD3 zeta chain fusion protein.

FIG. 3 depicts the use of a dimerizer binding to the extracellular portion of a protein to trigger intracellular signaling.

FIG. 4 depicts the use of a competitive binding assay to identify compounds which bind to a receptor protein.

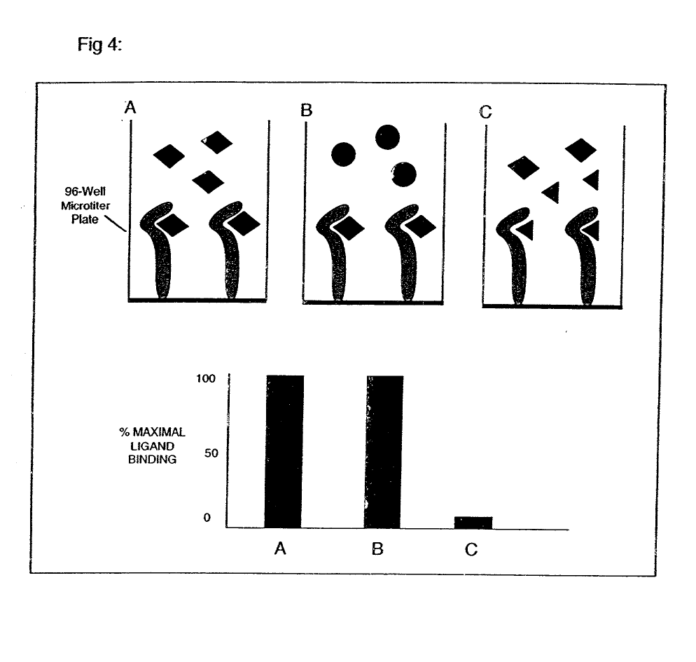

FIG. 5 depicts a screening assay to identify immobilized compounds which bind to a receptor protein.

FIG. 6 depicts EPO-induced signaling in cells expressing chimeric receptor proteins and the use of such systems to identify small molecule antagonists of EPO-binding.

FIG. 7 depicts a general methodology for the design and construction of an expression vector for producing a portion of a receptor protein, e.g., for use in binding experiments.

### DETAILED DESCRIPTION OF THE INVENTION

Dimerization: Generally

As noted above, this invention provides a generally applicable method and materials for utilizing homodimerization, heterodimerization and oligomerization of endogenous proteins in living cells to mediate a desired biological event. As noted above, the method uses dimerizers which are nonpeptidic, less than 5 kD in molecular weight and/or membrane permeant.

Homodimerization and homo-oligomerization refer to the association of like components to form dimers or oligomers, linked as they are by a dimerizer of this invention. Heterodimerization and hetero-oligomerization refer to the association of dissimilar components to form dimers or oligomers. Homo-oligomers thus comprise an association of multiple copies of a particular component while hetero-oligomers comprise an association of copies of different components. “Oligomerization”, “oligomerize” and “oligomer”, as the terms are used herein, with or without prefixes, are intended to encompass “dimerization”, “dimerize” and “dimer”, absent an explicit indication to the contrary.

Binding of the dimerizer to the receptor proteins hetero- or homodimerizes the proteins. Oligomerization brings the protein molecules into close proximity with one another thus triggering cellular processes normally associated the receptor protein-such as TCR-mediated signal transduction, for example.

Exemplary Receptor Proteins and Processes to be Triggered

Examples of protein mediators include transcription factors such as the STAT-91 protein and receptors for polypeptide growth factors and hormones such as those illustrated in Table I:

TABLE I

| Mediator | Receptor Type | Ligand | Therapeutic Appl’n |
|---|---|---|---|
| EPO receptor | Class I Cytokine | EPO | Anemia |
| G-CSF receptor | Class I Cytokine | G-CSF | Neutropenia |
| TPO receptor | Class I Cytokine | TPO | Thrombocytopenia |
| (c-Mpl) | | | |
| GH receptor | Class I Cytokine | GH | GH deficiency |
| IL-2 receptor | Class I Cytokine | IL-2 | Cancer |
| IFN-alpha receptor | Class II Cytokine | IFN-alpha | Hepatitis C |
| IFN-beta receptor | Class II Cytokine | lFN-beta | Multiple Sclerosis |
| Insulin receptor | Tyrosine kinase | Insulin | Diabetes |
| Trk receptors | Tyrosine kinase | NTFs | CNS diseases |

Polypeptide growth factors, cytokines and hormones, such as insulin, erythropoietin (EPO), growth hormone (GH) and granulocyte colony stimulating factor (G-CSF) activate intracellular processes upon binding to specific cell surface receptors (Ullrich, A. and Schlessinger, J., “Signal transduction by receptors with tyrosine kinase activity”, Cell 61: 203-212 (1990); Kishimoto, T., Taga, T., and Akira, S., “Cytokine signal transduction”, Cell, 76:253-262 (1994)). These receptors are composed of three domains: an extracellular ligand binding domain, a transmembrane domain, and an intracellular signal transduction domain. Some receptors, such as those for GH and EPO, have the ligand binding domain and signaling domain on the same polypeptide. Others, such as receptors for IL-3 and IL-6, have separate ligand binding and signal transduction subunits. It is now clear that signal transduction by cytokines and growth factors is accomplished by ligand-mediated receptor dimerization (Heldin, C. H., “Dimerization of cell surface receptors in signal transduction”, Cell. 80: 213-223 (1995); Lemmon, M. A., and Schlessinger, J., “Regulation of signal transduction and signal diversity by receptor oligomerization”, Trends Biol. Sci., 19:459-463 (1994)). For example, EGF binds to two receptor subunits resulting in dimerization of the cytoplasmic tyrosine kinase domains. This association of intracellular domains stimulates the tyrosine kinase activity and initiates a cascade of intracellular processes. Recent work on cytokine receptors has demonstrated that their signal transduction is also mediated by receptor dimerization (Murakami, M., et al, “IL-6-induced homodimerization of gpl 30 and associated activation of a tyrosine kinase”, Science, 260:1808-1810 (1993)).

Unlike G protein-coupled receptors where precise ligand-induced conformational changes are required for initiation of signaling events, receptors that are activated by dimerization require only imprecise aggregation of their cytoplasmic domains. For example, cells expressing an EPO receptor variant containing an additional extracellular cysteine were constitutively activated in the absence of EPO by the formation of disulfide linked receptor dimers (Watowich, et al, “Homodimerization and constitutive activation of the erythropoietin receptor”, Proc. Natl Acad. Sci. USA, 89:2140-2144 (1992)). In other studies, bivalent antibodies to growth hormone receptor dimerized the receptor subunits and activated GH-mediated cell proliferation (Fuh, G., et al, “Rational design of potent antagonists to the human growth hormone receptor”, Science, 256:1677-1680 (1992)).

Other signal-transducing proteins are noted in U.S. Pat. No. 5,830,462 (see e.g. col. 17, line 14 et seq). Many are tyrosine kinases or are complexed with tyrosine kinases, e.g. CD3 zeta, IL-2R, IL-3R, etc. For a review see Cantley, et al., Cell (1991) 64, 281. Tyrosine kinase receptors which are activated by cross-linking, e.g. dimerization (based on nomenclature first proposed by Yarden and Ulrich, Annu. Rev. Biochem. (1988) 57, 443, include subclass I: EGF-R, ATR2/neu, HER2/neu, HER3/c-erbB-3, Xmrk; subclass II: insulin-R, IGF-1-R [insulin-like growth factor receptor], IRR; subclass III: PDGF-R-A, PDGF-R-B, CSF-1-R (M-CSF/c-Fms), c-kit, STK-1/Flk-2; and subclass IV: FGF-R, fig [acidic FGF], bek [basic FGF]); neurotrophic tryosine kinases: Trk family, includes NGF-R, Ror1,2. Receptors which associate with tyrosine kinases upon cross-linking include the CD3 zeta family: CD3 zeta and CD3 eta (found primarily in T cells, associates with Fyn); beta and gamma chains of Fc_{ε}Rl (found primarily in mast cells and basophils); gamma chain of Fc_{γ}RIII/CD16 (found primarily in macrophages, neutrophils and natural killer cells); CD3 gamma, delat and epsilon (found primarily in T cells); Ig-alpha/MB-1 and lg-β/B29 (found primarily in B cell). Many cytokine and growth factor receptors associate with common β subunits which interact with tyrosine kinases and/or other signalling molecules and which can be used as cytoplasmic domains in chimeric proteins of this invention. These include (1) the common β subunit shared by the GM-CSF, IL-3 and IL-5 receptors; (2) the β chain gp130 associated with the IL-6, leukemia inhibitory factor (LIF), ciliary neurotrophic factor (CNTF), oncostatin M, and IL-11 receptors; (3) the IL-2 receptor g subunit associated also with receptors for IL-4, IL-7 and IL-13 (and possibly IL-9); and (4) the β chain of the IL-2 receptor which is homologous to the cytoplasmic domain of the G-CSF receptor.

The interferon family of receptors which include interferons alpha/β and gamma (which can activate one or more members of the JAK, Tyk family of tyrosine kinases) as well as the receptors for growth hormone, erythropoietin and prolactin (which also can activate JAK2) can also be used.

Othes include the TGF-β family of cell surface receptors (reviewed by Kingsley, D., Genes and Development 1994 8 133). This family of receptors contains serine/threonine kinase activity in their cytoplasmic domains, which are believed to be activated by crosslinking.

The tyrosine kinase receptors can be found on a wide variety of cells throughout the body. In contrast, the CD3 zeta family, the Ig family and the lymphokine β-chain receptor family are found primarily on hematopoietic cells, particularly T-cells, B-cells, mast cells, basophils, macrophages, neutrophils, and natural killer cells. The signals required for NF-AT transcription come primarily from the zeta chain of the antigen receptor and to a lesser extent CD3gamma, delta and epsilon.

The foregoing list is not exhaustive, but provides exemplary systems for use in the subject invention.

Additionally, note that cellular processes which can be triggered by oligomerization include a change in state, such as a physical state, e.g. conformational change, change in binding partner, cell death, initiation of transcription, channel opening, ion release, e.g. Ca⁺² etc. or a chemical state, such as a chemical reaction, e.g. acylation, methylation, hydrolysis, phosphorylation or dephosphorylation, change in redox state, rearrangement, or the like. Any such process which can be triggered by the association or oligomerization of endogenous cellular constituents is within the scope of this invention, including for example, signaling triggered by the association of mediators such as growth factor receptors and the “forwarding” of one endogenous constituent to the cellular environment or fate of another endogenous constituent using a dimerizing agent capable of binding to both constituents, Illustrative biological functions which can be controlled by oligomerization of proteins include protein kinase or phosphatase activity, reductase activity, cyclooxygenase activity, protease activity or any other enzymatic reaction dependent on subunit association, Also, one may provide for association of G proteins with a receptor protein associated with the cell cycle, e.g. cyclins and cdc kinases, multi-unit detoxifying enzymes.

Dimerizers: Generally

Generally speaking, the dimerizer is capable of binding to two (or more) protein molecules, in either order or simultaneously, preferably with a Kd value below about 10⁻⁶, more preferably below about 10⁻⁷, even more preferably below about 10⁻⁸, and in some embodiments below about 10⁻⁹ M. The dimerizer preferably is a non-protein and has a molecular weight of less than about 5 kDa. The proteins so oligomerized may be the same or different.

For binding to an intracellular domain of a protein, the dimerizer will be selected to be able to be transferred across the membrane in a bioactive form, that is, it will be membrane permeant. Various dimerizers are hydrophobic or can be made so by appropriate modification with lipophilic groups. Particularly, dimerizers containing linking moieties can be modified to enhance lipophilicity by including one or more aliphatic side chains of from about 12 to 24 carbon atoms in the linker moiety. Alternatively, one or more groups can be provided which will enhance transport across the membrane, desirably without endosome formation. In some applications, the dimerizers act extracellularly to bring together proteins which act in concert to initiate a physiological action. In such cases, the dimerizer need not necessarily be cell permeant.

In some instances, multimeric dimerizers need not be employed. For example, molecules can be employed where two different binding sites provide for dimerization of the receptor. In other instances, binding of the dimerizer can result in a conformational change of the receptor domain, resulting in activation, e.g. oligomerization, of the receptor. Other mechanisms may also be operative for inducing the signal, such as binding a single receptor with a change in conformation resulting in activation of the cytoplasmic domain.

Applicable and readily observable or measurable criteria for dimerizers include: (A) the dimerizer is physiologically acceptable (i.e., lacks undue toxicity towards the cell or animal for which it is to be used), (B) it has a reasonable therapeutic dosage range, (C) desirably (for applications in whole animals), it can be taken orally (is stable in the gastrointestinal system and absorbed into the vascular system), (D) it can cross the cellular and other membranes, as necessary, and (E) binds to the target protein(s) with reasonable affinity for the desired application. Preferably the dimerizer is relatively inert physiologically, but for its activating capability with the target protein(s).

Dimerizers: Homodimerization vs Heterodimerization

In embodiments in which the biological event of interest is mediated by association of two or more copies of the same mediator species, e.g. a receptor for a cytokine, growth factor or other hormone, the dimerizer is selected or designed for binding to multiple copies of the same protein mediator and may contain multiple copies of the same receptor binding moiety.

In embodiments in which the biological event of interest is mediated by association of two or more different mediator proteins, the dimerizer is selected or designed for binding to at least two different protein molecules and may contain two or more different receptor binding moieties. Examples of biological events which can be mediated by the association of different mediator proteins include transcriptional activation (mediated by association of a protein containing a DNA-binding domain with a protein containing a transcriptional activation domain), the targeting of a protein to a particular location (mediated by association of the protein to be targeted with a targeting protein), including the targeting of a protein for degradation via the proteosome, etc.

Dimerizers: Design of Multimeric Dimerizers

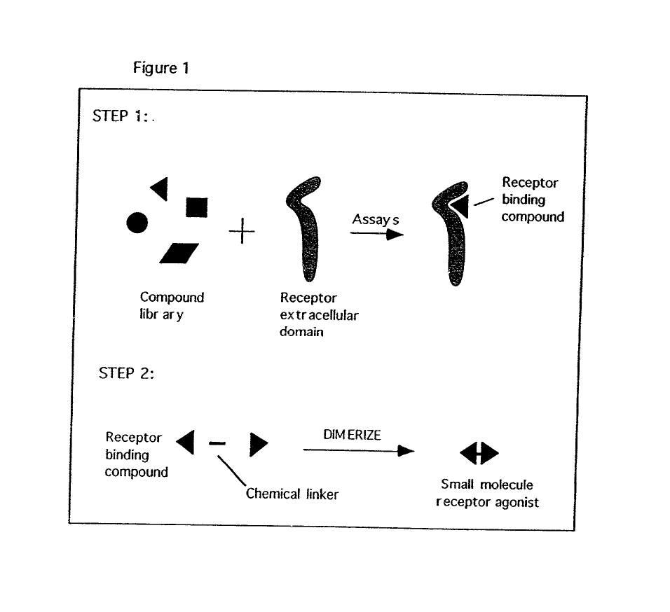

One method for preparing a dimerizer for use in this invention involves the steps of identifying a first compound capable of binding to one of the protein mediators and a second compound capable of binding to the other protein mediator. The two compounds are then covalently joined to one another to form a dimerizer which is capable of binding to both mediators (at the same time) as depicted schematically in FIG. 1. Methods are disclosed for identifying such monomeric binding compounds and for evaluating and optimizing the dimerizers produced from them.

Such dimerizers are molecules capable of binding to two or more protein molecules of to form an oligomer thereof, and have the formula: linker-{rbm₁, rbm₂, . . . rbmₙ}, wherein n is 2 or greater, rbm₍₁₎-rbm₍ₙ₎ are receptor binding moieties which may be the same or different and which are capable of binding to the relevant protein molecule(s). The rbm moieties are covalently attached to a linker moiety which is a bi- or multi-functional moiety capable covalently linking (“-”) two or more rbm moieties. Preferably the dimerizer has a molecular weight of less than about 5 kDa and is not a protein.

Such dimerizers are illustrated by compounds disclosed in U.S. Pat. No. 5,830,462 and include those in which the rbm moieties are the same or different and comprise an FK506-type moiety, a cyclosporin-type moiety, a steroid or tetracycline. Cyclosporin-type moieties include cyclosporin and derivatives thereof which are capable of binding to a cyclophilin, naturally occurring or modified, preferably with a Kd value below about 10⁻⁶ M. Illustrative dimerizers include those in which at least one rbm comprises a molecule of FK506, FK520, rapamycin or a derivative thereof. Linker moieties are also described in detail later, but for the sake of illustration, include such moieties as a C2-C20 alkylene, C4-C18 azalkylene, C6-C24 N-alkylene azalkylene, C6-C18 arylene, C8-C24 ardialkylene or C8-C36 bis-carboxamido alkylene moiety.

The monomeric rbm's of this invention, as well as compounds containing sole copies of an rbm, which are capable of binding to the relevant protein but not effecting dimerization or higher order oligomerization thereof (in view of the monomeric nature of the individual rbm) may be used as oligomerization antagonists.

Dimerizers: Choice of RBMs

Many compounds capable of binding to a variety of protein mediators of biological events are already known. For instance, many benzodiazepines, prostaglandins, beta-turn mimetics, alpha- and beta-blockers, FK506 (and related compounds such as rapamycin and their analogs), steroids, retinoids, topoisomerase inhibitors and other ligands which bind to their respective receptors or binding partners are known. Other compounds capable of binding to those receptors or to other endogenous constituents may be readily identified using a variety of approaches, including phage display and other biological approaches for identifying peptidyl binding compounds; synthetic diversity or combinatorial approaches (see e.g. Gordon etal, 1994, J Med Chem 37(9):1233-1251 and 37(10):1385-1401); and DeWitt et al, 1993, PNAS USA 90:6909-6913) and conventional screening or synthetic programs. Unlike programs to design or screen for biologically active compounds such as enzyme inhibitors or receptor agonists or antagonists, binding compounds for use in the subject invention may, but need not, bind to the mediator in a precise fashion required to inhibit, agonize or antagonize-they need only bind to the mediator. Compounds capable of binding to the protein of interest may be identified by various methods of affinity purification or by direct or competitive binding assays, including assays involving the binding of the protein to compounds immobilized on solid supports such as pins, beads, chips, etc.). See e.g. Gordon eta/, supra.

There are a variety of binding pairs of naturally-occurring receptors and small-molecule ligands which lend themself to the practice of this invention, Many such small molecule ligands will fulfill the desired binding criteria, and can be dimerized at various sites to provide a dimerizer according to the subject invention. Substantial modifications of these rbms are permitted, so long as the binding capability is retained and with the desired specificity. Suitable binding affinities will typically be reflected in Kd values well below 10⁻⁴, preferably below 10⁻⁶, more preferably below about 10⁻⁷, although binding affinities below 10⁻⁹ or 10⁻¹⁰ are possible, and in some cases will be most desirable.

Dimerizers: Assays for RBMs

For example, a known ligand for a receptor may be used as follows to identify compounds which bind to the ligand's receptor which may be used in dimerizers of this invention. Generically stated, the method of this embodiment employs: (1) a peptide which contains a ligand-binding domain of a receptor of interest (which may be intact receptor, the ligand-binding domain thereof or a fusion protein containing the ligand-binding domain of the receptor fused to heterologous protein sequence, collectively referred to as “receptor” in the following discussion), (2) a ligand for the receptor which is capable of selectively binding to the receptor to form a ligand-receptor complex and (3) a compound (the “test substance”) to be evaluated for its ability to bind competitively to the receptor. The method is carried out by combining the three components mentioned above, or compositions comprising them; incubating the resulting test mixture under conditions permitting the formation of a ligand-receptor complex; and measuring the ability of the test substance to compete with the ligand for binding to the receptor or to otherwise block the formation or reduce the observed level of receptor-ligand complex. This method is a powerful and general method, and should be applicable to any receptor-ligand pair and susceptible to variety of configurations, including both in vitro and in vivo formats. Depending on the specific assay configuration, it may be important to use known concentrations of receptor, ligand and/or test substance. For comparative purposes, the assay may also be carried out in the absence of the test substance or in the presence of varying concentrations of test substance. One may carry out the measuring step by assaying for receptor-ligand complex, non-complexed receptor and/or non/complexed test substance or by measuring the occurrence of an event mediated by the presence or formation of the receptor-ligand complex or a receptor-test substance complex. For example, in one embodiment, a ligand for a receptor is immobilized and incubated, under conditions permitting receptor-ligand binding, with a labeled receptor, or labeled peptide containing the ligand-binding domain of the receptor, in the presence and absence of a test substance or composition containing a test substance. The presence of a test substance which competes with ligand for receptor binding correlates with a decrease in labeled receptor (or labeled domain) bound to the immobilized ligand, or with an increase in unbound labeled receptor (or labeled domain). Various labels suitable for such purposes are well known in the art and may be selected based on factors such as cost, availability, convenience and familiarity on the part of the practitioner.

The test substance may be present in a solution, referred to as a test solution. Alternatively, especially for in vitro assays, the test substance may be present in a test mixture comprising an emulsion, suspension or other mixture; exposed on the surface of a cell, virus, phage, etc.; or immobilized on a solid support.

In an in vitro format, a binding assay is conducted to identify a compound capable of binding to the receptor in the presence of a ligand for that receptor or otherwise capable of blocking the formation or reducing the observed level of receptor-ligand complex. In one embodiment, the binding assay is a competitive binding assay in which the three components are combined and incubated under conditions permitting the formation of an receptor-ligand complex. The ability of the test substance to bind to the selected receptor or otherwise block the receptor-mediated interaction in the presence of the receptor's ligand is determined.

Binding to the receptor or otherwise blocking the receptor-mediated interaction may be measured directly or indirectly (e.g., BIAcore® and other SPR technologies (BIAtechnology Handbook, Pharmacia Biosensor AB, Uppsala, Sweden, 1994), fluorescence anisotropy and allied technologies (Luminescent Spectroscopy of Proteins, 164 pp, E. A. Permyakov, CRC Press, Inc, Boca Raton, Fla., 1992), flow cytometry and allied technologies (Flow Cytometry and Cell Sorting, 223 pp., A. Radbruch, ed., Springer-Verlag, New York, N.Y., 1992), ELISA, RIA and allied methodologies (An Introduction to Radioimmunoassays and Related Techniques, 290 pp., T. Chard, Elsevier Science Publishers, Amsterdam, The Netherlands, 1990), competitive and non-competitive affinity interactions (Immobilized Affinity Ligand Techniques, 454 pp., G. T. Hermanson, A. K. Mallia and P. K. Smith, eds., Academic Press, Inc., San Diego, Calif., 1992).

In competitive binding assays, if binding of the receptor and its ligand occurs to a lesser extent in the presence of the test substance than in its absence, for instance, if the presence of the test substance reduces the concentration of receptor-ligand complex or increases the concentration of non-complexed (i.e., to each other) receptor or ligand, then the test substance is a receptor-binding agent. If the structure of the binding agent so identified is not yet known, the compound may then be isolated from the other assay components and characterized. It may be re-evaluated, if desired, using similar binding assays with different receptor-ligand pairs to confirm the selectivity of the interaction with the receptor with which it was identified. If desired, the binding of the binding agent to the receptor with which it was identified may be characterized biochemically, e.g. through the use of BIAcore® technology, described in greater detail below. The binding agent so identified may be assayed in an in vivo assay as described below and may further be evaluated in monomer and/or dimerizer form for pharmacological activity in various in vitro and/or in vivo assays, as desired.

In vivo assays can be conducted in analogous manner using cells containing the ligand-binding domain of interest and a ligand therefor. The cells are cultured or maintained in a medium suitable for cell growth. The test substance is added to the cells, e.g. to the medium in which the cells are cultured, and the culture is incubated under conditions permitting formation of a complex between the receptor and its ligand. If binding of the receptor and its ligand occurs to a lesser extent in the presence of the test substance than in its absence, for instance, if the presence of the test substance reduces the concentration of receptor-ligand complex or increases the concentration of non-complexed ligand, then the test substance is an binding agent. The presence or absence of receptor-ligand complex may be measured directly or indirectly (e.g., by measuring the occurrence of an event mediated by the presence or formation of the receptor-ligand complex or a receptor-test substance complex).

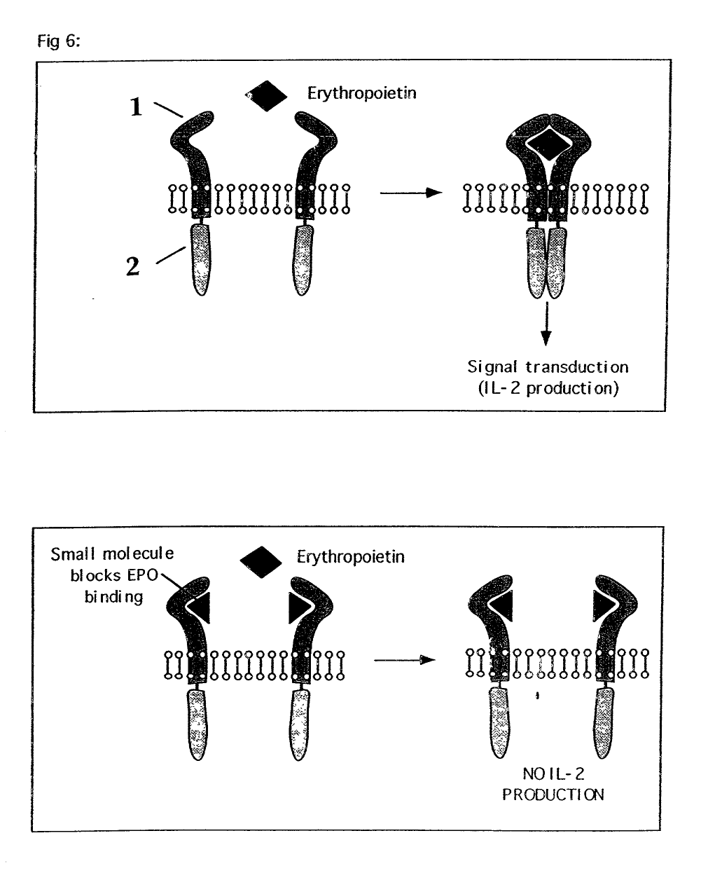

An illustrative in vivo format relies upon genetically engineered cells capable of expressing a reporter gene under receptor-mediated transcriptional control. These cells contain and are capable of expressing recombinant DNAs encoding a fusion protein comprising, among other component regions, at least one ligand-binding domain of the receptor of interest. The fusion proteins are capable of binding to the ligand for the receptor and in the presence of the ligand are capable of forming a complex (dimerizing) with each other as illustrated in FIG. 6. In the presence of the ligand, e.g. when maintained in culture medium containing ligand, the cells express the reporter gene-unless a substance is present which binds to the receptor domain or otherwise blocks the association of the fusion proteins required for transcription of the reporter gene. In this assay, the cells are cultured or maintained in a suitable culture medium to which a selected amount of ligand is added to establish a base-line for expression of the reporter gene. The test substance is then added to the culture medium and the ability of the test substance to inhibit expression of the reporter gene is measured. If the level of reporter gene expression is reduced in the presence of the test substance, the test substance is a blocker with respect to the chimeric receptor molecules involved in transcriptional control. If the structure of the blocking agent so identified is not yet known, the compound may then be isolated from the other assay components and characterized. It may be re-evaluated, if desired, using engineered cells containing a fusion protein based on a different receptor, in medium containing ligand for that receptor, to confirm the selectivity of the interaction with the receptor domain with which it was identified. If desired, the binding affinity of the blocking agent for the receptor with which it was identified may be determined, e.g. such as through the use of BIAcore® technology. The blocking agent so identified may be assayed in an in vitro binding assay as described above and may further be evaluated for pharmacological activity in various in vitro and/or in vivo assays, as desired, again in monomer and/or dimerizer (i.e., dimerized) form.

Binding agents identified by such methods for use in constructing dimerizers of this invention can be identified from peptide libraries as well as from test substances obtained from a wide variety of sources including, e.g., microbial broths; cellular extracts; conditioned media from cell lines or from host cells transformed with genetic libraries; collections of synthetic compounds; combinatorial libraries or synthetic programs based on conventional medicinal chemistry approaches or structure-based drug design.

By these and other means the practitioner can readily identify selective binding or blocking agents. Compounds so identified may be covalently joined together using linker moieties, e.g. by adaptation of the approaches disclosed in U.S. Pat. No. 5,830,462 and International Patent Application PCT/US94/08008, to form the dimerizers of this invention. Linker moieties need not contain essential elements for binding to the mediators of interest, and may be selected from a very broad range of structural types.

Linkers

Various linking groups can be employed, usually of from 1-30, more usually from about 1-20 atoms in the chain between the two molecules (other than hydrogen), where the linking groups will be primarily composed of carbon, hydrogen, nitrogen, oxygen, sulphur and phosphorous. The linking groups can include a wide variety of functionalities, such as amides and esters, both organic and inorganic, amines, ethers, thioethers, disulfides, quaternary ammonium salts, hydrazines, etc. It can include aliphatic, alicyclic, aromatic or heterocyclic groups. The linking moiety will be selected based on ease of synthesis and the stability of the multimeric ligand. Thus, if one wishes to maintain long-term activity, a relatively inert chain will be used, so that the multimeric ligand link will be resistant to cleavage. Alternatively, if one wishes only a short half-life in the blood stream, then various groups can be employed which are readily cleaved, such as certain esters and amides, particularly peptides, where circulating and/or intracellular proteases can cleave the linking group.

Illustrative linker moieties for use in dimeric or multimeric dimerizers include C₂-C₂₀ alkyl, aryl, or dialkylaryl structures. Other such linking moieties commonly include alkylene, usually of from 2 to 20 carbon atoms, azalkylene (where the nitrogen will usually be between two carbon atoms), usually of from 4 to 18 carbon atoms), N-alkylene azalkylene (see above), usually of from 6 to 24 carbon atoms, arylene, usually of from 6 to 18 carbon atoms, ardialkylene, usually of from 8 to 24 carbon atoms, bis-carboxamido alkylene of from about 8 to 36 carbon atoms, etc. Illustrative groups include decylene, octadecylene, 3-azapentylene, 5-azadecylene, N-butylene 5-azanonylene, phenylene, xylylene, p-dipropylenebenzene, bis-benzoyl 1,8-diaminooctane and the like. Multivalent or other (see below) ligand molecules containing linker moieties as described above can be evaluated using materials and methods such as described herein.

The multimeric ligands can be synthesized by any convenient means, where the linking group will be at a site which does not interfere with the binding of the binding site of a ligand to the receptor. Where the active site for physiological activity and binding site of a ligand to the receptor domain are different, it will usually be desirable to link at the active site to inactivate the ligand.

Alkyl is intended to include both saturated and unsaturated straight chain, branched, cyclic, or polycyclic aliphatic hydrocarbons which may contain oxygen, sulfur, or nitrogen in place of one or more carbon atoms, and which are optionally substituted with one or more functional groups selected from the group consisting of hydroxy, C₁-C₈ alkoxy, acyloxy, carbamoyl, amino, N-acylamino, ketone, halogen, cyano, carboxyl, and aryl (unless otherwise specified, the alkyl, alkoxy and acyl groups preferably contain 1-6 contiguous aliphatic carbon atoms).

Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry).

Linker moieties may be conveniently joined to the rbms through functional groups such as ethers, amides, ureas, carbamates, and esters; or through alkyl-alkyl, alkyl-aryl, or aryl-aryl carbon-carbon bonds. Furthermore, linker moieties may be optimized (e.g., by modification of chain length and/or substituents) to enhance pharmacokinetic properties of the multimerizing agent. In cases in which the compounds are identified while immobilized, they may be conveniently linked using the functional groups by which they had been immobilized. Peptidyl compounds may be linked by peptide bonds, although the preferred agents of this invention are not polypeptides or oligopeptides. Divalent dimerizing agents of this invention retain binding capability with respect to both of the proteins of interest. Thus, the covalently linked dimerizers will usually be tested (e.g. as above) to confirm retention of binding capability with respect to each of the proteins of interest and/or in cell-based assays as described below.

We note that in the design of dimerizers, selecting compounds which bind to the proteins of interest from combinatorial libraries immobilized on solid supports such as beads provides a useful advantage. While this still entails identifying a new ligand for the protein of interest rather than selecting a previously known compound, compounds so identified are identified together with an attachment point and chemistry for the design and/or assembly of dimerizers. That is so because the members of an immobilized combinatorial library are already covalently linked to their support. Thus, the same chemistry may be used in assembling the dimerizer as was used to immobilize the individual members of the library. Furthermore, since the selected library members are selected for their ability to bind to their respective receptors (or other protein binding partners), by necessity, the linker covalently attaching the library member to the support must not interfere with the binding interaction between the library member and the protein of interest.

Assays for Functional Evaluation of Dimerizers

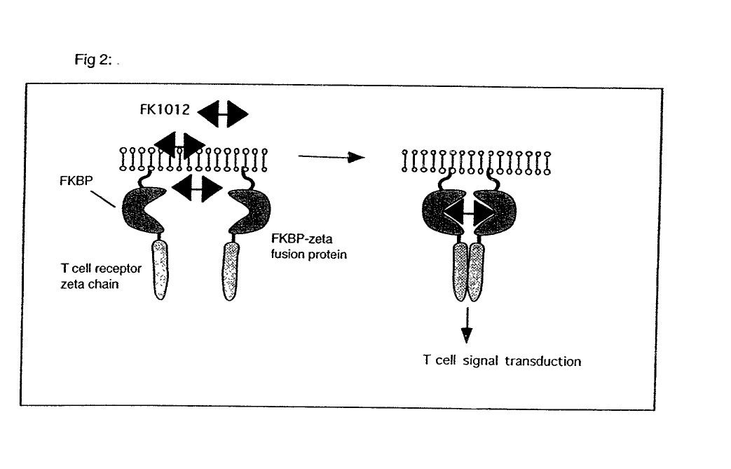

We also provide general cell-based assay methods for functional characterization of dimerizers. These assays are based on cells genetically engineered in accordance with the system described in U.S. Pat. No. 5,830,462. The cells are engineered to contain and be capable of expressing recombinant DNAs encoding chimeric proteins capable, upon their association or dimerization, of activating the transcription, directly or indirectly, of a reporter gene under the transcriptional control of a promoter, enhancer or other transcriptional regulatory element, responsive to the association of the chimeric proteins. Suitable materials, methods and design and construction principles for relevant constructs and their use are disclosed in U.S. Pat. No. 5,830,462 and may be adapted for use in the practice of this invention as illustrated by the following example. In one embodiment, Jurkat cells are genetically engineered to contain a reporter gene such as secreted alkaline phosphatase (although any conveniently detected reporter may be used, including beta-galactosidase or luciferase for example) under the expression control of the NF-AT system, details for which are provided in the above-mentioned patent. Those cells disclosed in the U.S. Pat. No. 5,830,462 were further engineered to contain and express a recombinant DNA sequence encoding a chimeric protein comprising a myristoylation signal, the cytoplasmic tail of the zeta chain of the T cell receptor and one or more ligand-binding domains derived from FKBP12. The cells of our assay are prepared analogously, but express one or more chimeric proteins containing, in place of the FKBP12 domain, part or all of the protein mediator of interest. Where dimerization of two different protein mediators is of interest, the cells are engineered to express a chimeric protein, as described above, corresponding to each protein mediator of interest. The presence of a dimerizer which is capable of binding to two molecules of the chimeric protein(s) induces association or dimerization of the chimeric proteins. Such dimerization triggers a transcriptional activation signal which is received by the transcriptional control elements for the reporter gene and is readily detected by measuring the expression of the reporter molecule as depicted schematically in FIG. 2. Full details and general guidance for assembling such constructs, engineering the cells and detecting the reporter molecule are provided in U.S. Pat. No. 5,830,462. See e.g. FIGS. 14, 15, and 18-21 and corresponding examples therein. Again, adapting that system to provide cells for assaying dimerizers of this invention is readily accomplished by replacing DNA sequence(s) encoding the FKBP domains with DNA sequence encoding the protein mediator of interest (for example the intracellular or extracellular domain of the receptor for insulin or erythropoietin, or other growth factor), taking care that the full coding region of each resultant construct is in frame.

Using such engineered cells one may functionally characterize dimerizers of this invention by growing the engineered cells in culture, exposing them to the dimerizer(s) of interest by adding an amount (usually a predetermined amount) of the dimerizer of interest to the culture medium, and detecting the amount of reporter produced in response to the dimerizer. Candidate dimerizers containing one or more structural variations in their component binding moieties or linking moiety may be comparatively evaluated and dimerizers for particular applications may thus be optimized.

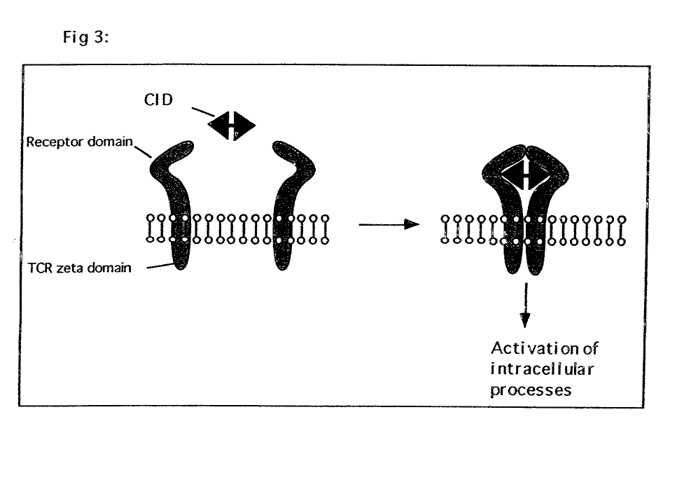

As an alternative approach to genetically engineered cells for such assay purposes, one may use a modified design for the chimeric “receptors” which will bind to the dimerizers and trigger transcription of the reporter gene. In this approach the chimeric receptors contain a signaling moiety such as the zeta chain as above, a membrane spanning domain, and, as an extracellular domain, one or more copies of a domain corresponding to the protein mediator of interest of the chimeric protein(s). The assay may be conducted as described above. However, in this modification, dimerizers are detected extracellularly rather than intracellularly, as depicted schematically in FIG. 3. This approach will usually be preferred for evaluating peptidyl or other dimerizers which do not readily enter the cells.

Dimerizers: Examples of Immunophilin-Based Dimerizers

Illustrative monomeric, dimeric and trimeric compounds based on rbms which bind to immunophilin or cyclophilin proteins are depicted below. The design, synthesis and use of these compounds is disclosed in detail in U.S. Pat. No. 5,830,462. A variety of other dimerizers are further disclosed in the Examples which follow and in International Patent Application PCT/US95/14177 and U.S. Ser. No. 08/973,337 (filed Nov. 18, 1997), Ser. No. 08/332,995 (filed Nov. 1, 1994), Ser. No. 08/400,800 (filed Mar. 7, 1995) and Ser. No. 08/480,286 (filed Jun. 7, 1995).

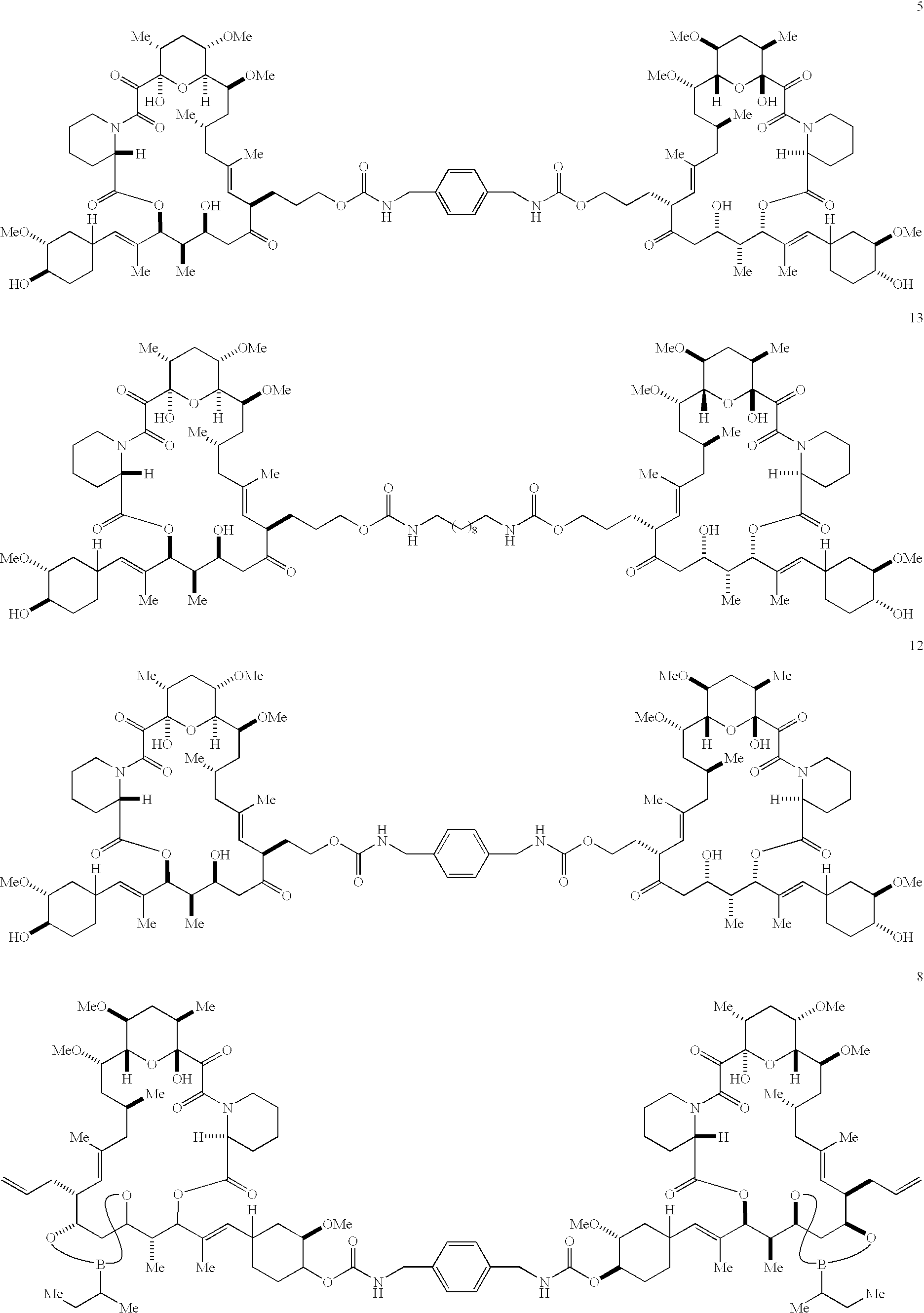

Administration of Dimerizers

Where the protein mediators of the biological event of interest are present on or within a cell, contacting such cells with an amount of the dimerizer effective to result in association of the mediator protein(s) results in the occurrence of the biological event of interest, e.g. in gene transcription, protein localization, receptor signalling, etc. Contacting the cells with the dimerizing agent is effected by adding the dimerizer to the culture medium in which the cells are growing, or, if the cells are or may be present within an organism, by administration of the dimerizer to the organism. The organism may be plant or animal, and in the latter case may be an insect, mammal (including among others, rodents such as mice and rats, and primates, including humans) or other animal. In cases in which the dimerizer is administered to an animal or human, it may be administered in the form of a veterinary or pharmaceutical composition containing the dimerizer and one or more suitable diluents, carriers, adjuvants and the like, as are well known in the art. Such compositions may contain conventional carriers for the various modes of administration including oral and parenteral administration.

The dimerizer may then be administered as desired. Depending upon the binding affinity of the dimerizer for the relevant protein molecule(s), the response desired, the manner of administration, the half-life of the dimerizer, the number of cells and receptor protein molecules/cell, various protocols may be employed. The dimerizer may be administered parenterally or orally. The number of administrations will depend upon factors such as described above. The dimerizer may be taken orally as a pill, powder, or dispersion; bucally; sublingually; injected intravascularly, intraperitoneally, subcutaneously; by inhalation, or the like. The dimerizer (and monomeric compound) may be formulated using conventional methods and materials well known in the art for the various routes of administration. The precise dose and particular method of administration will depend upon the above factors and be determined by the attending physician or human or animal healthcare provider. For the most part, the manner of administration will be determined empirically.

In the event that the action triggered by the dimerizer is to be reversed, a corresponding monomeric compound (or other single binding site compound which can compete with the dimerizer) may be administered. Thus, in the case of an adverse reaction or the desire to terminate the therapeutic effect, the monomeric binding compound can be administered in any convenient way, particularly intravascularly, if a rapid reversal is desired.

The particular dosage of the dimerizer for any application may be determined in accordance with the procedures used for therapeutic dosage monitoring, where maintenance of a particular level of pharmacologic result is desired over an extended period of times, for example, greater than about two weeks, or where there is repetitive therapy, with individual or repeated doses of dimerizer over short periods of time, with extended intervals, for example, two weeks or more. A dose of the dimerizer within a predetermined range would be given and monitored for response, so as to determine a dose-response relationship over a time period, as well as observing therapeutic response. Depending on the levels observed during the time period and the therapeutic response, one could provide a larger or smaller dose the next time, following the response. This process would be iteratively repeated until one obtained a dosage within the therapeutic range. Where the dimerizer is chronically administered, once the maintenance dosage of the dimerizer is determined, one could then assay at extended intervals to be assured that the cellular system is providing the appropriate pharmacologic response.

It should be appreciated that the system is subject to many variables, such as the cellular response to the dimerizer, the particular need of the patient (which may vary with time and circumstances), and the like. Therefore, it is expected that proper dosage level may be optimized for particular indications and for individual patients.

Uses

As mentioned at the outset, a wide variety of receptor/ligand pairs are involved in a number of pharmacologically significant events including anemia, neutropenia, thrombocytopenia, cancer, MS, diabetes, CNS disorders, etc and their treatment. Accordingly, dimerizers of this invention may be useful for a variety of clinically important purpose as well as for research purposes to probe the biology of receptor-mediated phenomena.

Generally speaking, dimerizers of this invention can be used to promote the occurrence of biological events resulting from molecular interactions mediated by a receptor of interest. This invention thus provides a method and reagents for promoting the interaction between endogenous proteins and thus for promoting a biological activity mediated by such interaction. In this method, a dimerizer of this invention is combined or contacted with the receptor of interest, such as by introducing the dimerizer into a cell in which the receptor-mediated interaction is to be promoted. Following introduction of the dimerizer, the mutual association or dimerization of the endogenous protein to which the dimerizer binds is promoted, as may be readily detected. Promoting such interactions can be useful in research aimed at better understanding the biology of receptor-mediated events.

Such dimerizers would be useful, for example, in the diagnosis, prevention or treatment of conditions or diseases which may be cured, or have their symptoms alleviated in whole or part, by the occurrence of cellular processes mediated by a receptor-mediated interaction. For example, a patient can be treated to prevent or alleviate the occurrence or progression of anemia, thrombocytopenia, or neutropenia by the administration of a dimerizer capable of promoting dimerization of receptor molecules for EPO, TPO or G-CSF, respectively.

A dimerizer of this invention can be formulated into a pharmaceutical composition containing a pharmaceutically acceptable carrier and/or other excipient(s) using conventional materials and means. Such a composition can be administered to an animal, either human or non-human, for therapy of a disease or condition responsive to the promotion of cellular events involving the mutual interaction of endogenous protein molecules. Administration of such composition may be by any conventional route (parenteral, oral, inhalation, and the like) using appropriate formulations as are well known in this art. The dimerizer can be employed in admixture with conventional excipients, i.e., pharmaceutically acceptable organic or inorganic carrier substances suitable for parenteral administration.

Equivalents

The full contents of all references cited in this document, including publications from the scientific literature, issued patents and published patent applications, are hereby expressly incorporated by reference.

The following examples contain important additional information, exemplification and guidance which can be adapted to the practice of this invention in its various embodiments and the equivalents thereof. The examples and other illustrative embodiments provided herein are offered by way of illustration only and should not be construed as limiting in any way. As noted throughout this document, the invention is broadly applicable and permits a wide range of design choices by the practitioner.

### EXAMPLES

### Example 1

### Enhancing the Activity of Known Drugs or Newly Selected Compounds, or Imparting an Activity, by Incorporation into a Dimerizer

In this approach, a protein which functions substantially in only one cellular compartment, e.g. the cytoplasm, is diverted through binding to an appropriate dimerizer to an alternative cellular compartment where it lacks bioactivity.

To design such a dimerizer, one selects a first compound capable of binding to the protein target. Examples of protein targets that function only in the cytoplasm and not in the nucleus, for example, include HIV protease and various signaling proteins such as zap70, syk and the like. In the case of HIV protease, cell permeant HIV protease inhibitors have been developed by a number of groups and are known in the literature. See e.g. Lam et al, 1994, Science 263(5145): 380-4 and International Patent Application WO 94/08977.

One further selects a second compound which binds to a constituent of the alternative cellular compartment. Again, localization of the target protein in the alternative compartment is inconsistent with biological function of the target protein. Where the alternative compartment is the nucleus, relevant constituents include the topoisomerases to which etoposide, camptothecin and related compounds bind. The synthesis of Camptothecin and analogs thereof is known, as is their evaluation as inhibitors of topoisomerase I. See e.g. Corey et al, 1975, J Org Chem 40:2140 (total synthesis); Sugimori et al, 1994, J. Med. Chem. 37(19), 3033-9 (illustrative analogs); Prost et al, 1994, Biochem. Pharmacol. 48(5), 975-84 (experiments with topo 1). Alternate nuclear targets include DNA, for which numerous intercalating agents are known. Alternative targets for directing a target protein to the mitochondria include cytochromes which are present only in that compartment.

The first and second compounds may be selected from known compounds capable of binding to the respective proteins or may be selected from combinatorial libraries as discussed above. In either event, they are then covalently joined through a linker moiety as mentioned above in a way which does not abrogate either of the individual binding interactions (ie, to either of the two proteins). The resultant dimerizer can be readily evaluated to confirm retention of suitable binding behavior.

To illustrate this approach we have designed a dimerizer based on an HIV protease inhibitor and a camptothecin analog to bind to the HIV protease and translocate it into the nucleus. The incorrect compartmentalization of the protease resulting from the translocation is aimed at effectively inactivating the HIV protease. Such dimerizers may bind to the protease active site and inhibit its enzymatic activity as do other HIV protease inhibitor molecules. But whether they do so or not, these dimerizers are designed to abrogate protease activity by translocation to an incorrect cellular compartment.

In our illustrative approach, S-10-hydroxycamptothecin (3) and other analogs of camptothecin are obtained by known procedures. See e.g. Kingsbury et al, 1991, J. Med. Chem., 34(1), 98-107. See also, Luzzio et al, Eur. Pat. AppI. EP 540099.

Hydroxycamptothecin may be linked to an HIV protease inhibitor (2)(see Lam et al and WO 94/08977, both supra, to form the dimerizer (1)

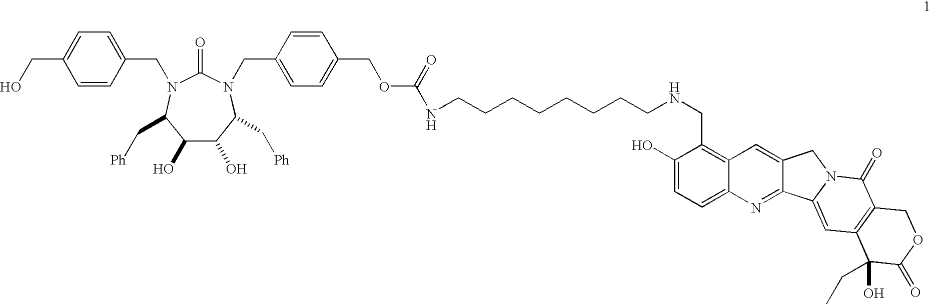

as depicted schematically below (see Kingsbury et al):

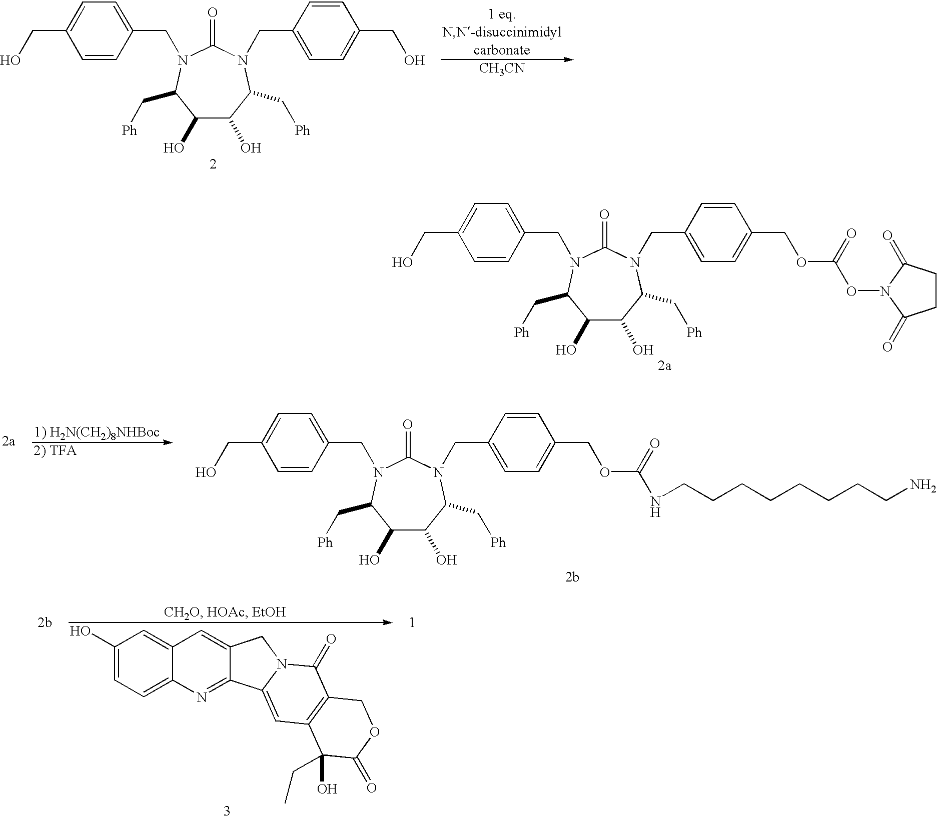

### Example 2

The approach of Example 1 is extendable to other cytoplasmic targets, e.g. zap70, to illustrate the targeting of a signal transduction mediator. While some groups are actively searching for compounds which bind to and specifically inhibit zap70, a dimerizer of this invention which contains a zap70 binding molecule (even one which alone does not inhibit zap70 interactions or biological activity) linked to a nuclear targeting moiety such as a camptothecin moiety or the like or a protesome targeting moiety (see below) would translocate the target to an incorrect compartment, i.e. a cellular location, inconsistent with its normal biological functioning, or to the proteosome where it can be removed from the system by degradation.

### Example 3

An additional illustrative example involves the targeting of a cellular protein or component of virus such as HIV to proteosomal degradation pathways using dimerizers. These dimerizers have as one component, molecules known (or selected) to bind to viral proteins such as AZT and as a second component a molecule that binds to proteosome components such as the LMP7, LMP2 (Martinez and Monoco, Nature 353:664, 1991) or other components responsible at least in part for proteosome function and substrate specificity (Gaszynska, M. et al Nature 365,264,1993; A. Skiyama et al FEBS-Lett.343: 85-88,1994; and Shimbara et. al. J Biochem 115:257,1994). As in the other embodiments of this invention the binding compounds may be selected from previously known compounds which are known or thought to possess the desired binding properties, or may be selected using conventional or other binding assays from collections of compounds screened against the protein of interest (expressed for instance using conventional methods and materials). These dimerizers are designed to induce the physical proximity of the targeted viral or cellular proteins to the proteosome, thereby resulting in the rapid destruction of the cellular or viral protein.

Thus, molecules that bind to the proteosome may be identified by screening of collections of compounds or by a variety of methods described above. Compounds may also be so selected from combinatorial libraries or from the store of previously known compounds which are capable of binding to essential HIV proteins, including AZT and analogs thereof and any of the numerous reported molecules that bind to the HIV protease. A compound which binds to a proteosome component is then covalently linked to one of the compounds capable of binding to the targeted HIV component.

The efficiency of the dimerizers in inducing dimerization may be tested using the cell based assay described above. This method allows for comparative evaluation of modifications in dimerizer design, including modifications to binding molecules, linker moiety and specific linkages. Following confirmation of desired activity in the induction of dimerization and activation of the zeta chain chimera, the dimerizers may be tested for the intended biological activity, e.g. the ability to rid cultured cells of the target protein, using assays such as western blotting and other established methods or bioassays.

### Example 4

A further illustrative modification involves selecting a compound capable of binding to a class of proteins called E3 enzymes (see Ciechanover, 1994, Cell 79:13-21 and references cited therein). These cause proteins to which they bind to be ubiquitinated and therefore targeted for protein degradation. An example of a protein that acts by this principle is the E6 protein of the papilloma virus. It binds to an E3 protein called E6AP (E6-associated protein). E6 also binds to p53, p53 being brought in the close proximity of E6AP and E3 ligase causes it to be ubiquitinated and therefore degraded.

Dimerizers of this aspect of the invention are designed to contain a moiety selected for its ability to bind to an E3 enzyme covalently linked to a moiety selected to bind to a cellular, viral or other protein to be removed (e.g. HIV protease, zap 70, etc.). Binding of the dimerizer to the targeted protein is intended to result in ubiquitination and therefore degradation of the targeted protein by the cell.

### Example 5

### Iin vitro Competitive Binding Assay for Binding Compounds for use in Preparing Dimerizers

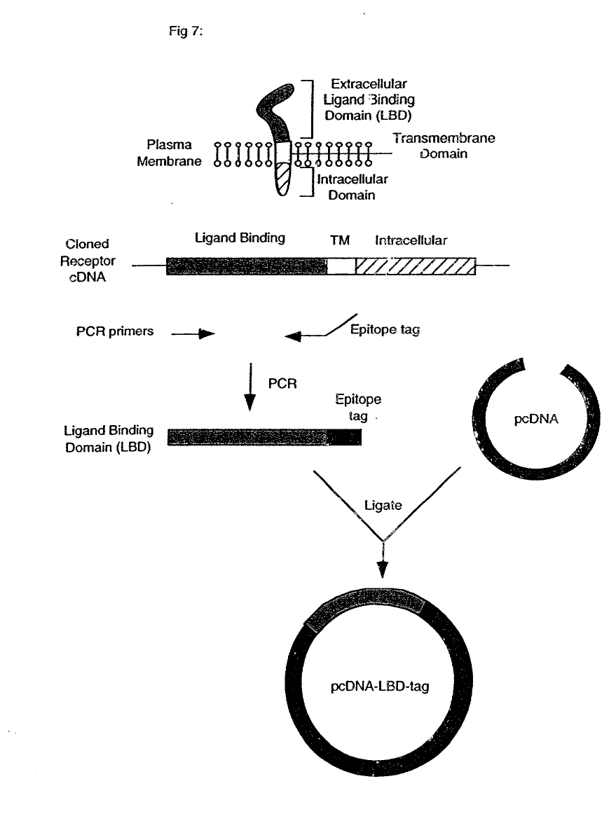

The extracellular ligand binding domain may be expressed and purified using the cloned receptor cDNA. Identification of the receptor extracellular domain can be done by performing a Kyte-Doolittle analysis on the coding sequence. In the case of cytokine and growth factor receptors, the extracellular domain is N-terminal of the transmembrane-spanning (TM) domain. The TM domain marks the end of the ligand binding domain and in the Kyte-Doolittle profile is demarked by a high hydrophobicity index over a span of between 20-30 amino acids. For an example of the Kyte-Doolittle analysis of the EPO-receptor see U.S. Pat. No. 5,278,065. See also U.S. Pat. No. 5,292,654 (mutant EPO-R). To produce the ligand binding domain of a receptor, the cDNA encoding the extracellular domain is cloned into an appropriate expression vector such as pET11 a (Invitrogen) for E. coli, pVL1393 (Invitrogen) for insect cells, or pcDNA (Invitrogen) for mammalian cells. A stop codon is introduced at/before the first amino acid of the TM domain. When this so-called soluble receptor is expressed in yeast, insect cells or mammalian cells, the protein is secreted into the cell culture medium (see Kikuchi et al J. Immunol. Methods 167:289 1994). Alternatively, when the ligand binding domain is expressed in E. coli, the soluble receptor collects in the periplasmic space (see Cunningham et al Science 254: 821 1991). To facilitate purification and binding assays the extracellular domain may be expressed fused to an epitope tag such as the epitope for the anti-myc antibody 9E10 or the “Flag” epitope (IBI) (see Kolodziej and Young, Methods Enzymol 194: 508 (1991)), Alternatively, the ligand binding domain may be expressed fused to the heavy chain of an immunoglobulin as described in (Ashkenazi et al PNAS 88:10535 1991). The ligand binding domain can be expressed in E. coli, yeast, insect cells, mammalian cells or produced using an in vitro transcription/translation system (Promega). Expression in mammalian cells can be accomplished using transient expression or by stable selection of clones using a selectable drug such as G418. For details of expression systems see Goeddel (ed.) Methods Enzymol vol 185 1990). See also, FIG. 7.

Purification of the expressed protein can be accomplished by standard chromatographic methods, by ligand affinity chromatography or by means of the fusion partner such as an antibody epitope or immunoglobulin heavy chain.

To assay for compounds that block ligand-receptor interactions, the purified ligand binding domain is first immobilized in a microtiter dish and mixed with a test compound and radiolabeled-ligand. Typically the ligand is iodinated such as in Pennica et al Biochemistry 31:1134 1992. After a suitable incubation time, the wells are washed with buffer and the bound ligand is determined by scintillation or gamma counting. Compounds that interfere with binding of the ligand are detected by a reduction in radioactivity bound to the plate. The ligand binding domain can be engineered to facilitate several aspects of the assay. For example, if the receptor ligand binding domain is expressed as a fusion protein to an immunoglobulin heavy chain, the protein can be bound to the microtiter plate via an antibody to the heavy chain constant region. Alternatively, the assay can be done in solution then the bound and unbound ligand separated by immunoprecipitation using protein-A sepharose or Pansorbin (Calbiochem) see Pennica et al Biochemistry 31:1134 1992. In addition, amino acid substitutions can be introduced into the ligand to prevent dimerization of the receptor (see Fuh et al Science 256:1677 1992). This will make it easier to detect organic small molecules that interfere with ligand binding.

Other binding assay configurations may be advantageous. For example one may attach the ligand to a plate and then incubate the test compound in solution with the soluble receptor. After a suitable time, the wells are washed and the amount bound receptor is detected. Detection can be afforded by direct radiolabeling of the receptor or via some tag on the receptor. For example, ELISA through an epitope tag, ELISA via a non-interfering epitope of the receptor or via biotin that was used to label the receptor. An alternative assay utilizes the BIAcore where the ligand is immobilized on the flow cell (“chip”) and binding of ligand in the absence or presence of test compound is measured (see Corcoran et al Eur. J. Biochem. 223:831 1994).

### Example 6

### Identification of Receptor Binding Molecules from Synthetic Molecular Diversity Libraries

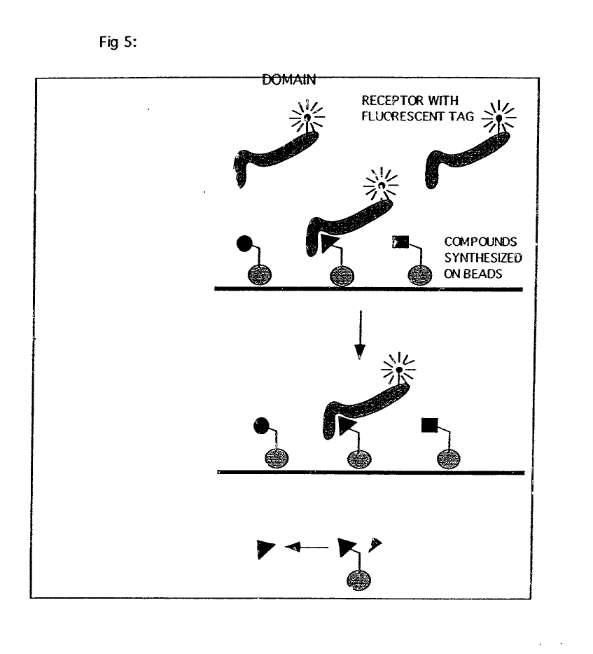

Novel ligands may also be identified using synthetic combinatorial libraries immobilized on beads, (Gordon, E. M., Barrett, R. W., Dower, W. J., Fodor, S. P. and Gallop, M. A., “Applications of combinatorial technologies to drug discovery. 2. Combinatorial organic synthesis, library screening strategies, and future directions”, J. Med. Chem., 37:1385-1401 (1994)) each of which contains a unique compound. Using known methods and materials, one can synthesize libraries of millions of peptide and non-peptide ligands. To screen the library, the purified receptor extracellular domain is labeled then incubated with the beads in an appropriate buffer. After washing the mixture, beads that have bound the receptor are identified. The selected beads are isolated, and the structure of the compound on the bead is determined (See FIG. 5). Various materials and methods are known in the art which are suitable for labeling the receptor so that the bound bead can be detected. These include labeling the receptor using a fluorescent molecule, biotin or an epitope tag fused to the domain. Visualization can be accomplished by fluorescence microscopy or an enzyme-linked assay using a substrate that makes the bead with bound receptor observable. Immobilized combinatorial libraries have been used, for instance, to identify ligands that bind to the Src SH3 domain (Yu, H, Chen, J. K., Feng, S, Dalgarno, D. C., Brauer, A. W., and Schreiber, S. L., “Structural basis for the binding of proline-rich peptides to SH3 domains”, Cell, 76:933-945 (1994)).

### Example 7

### Antagonism of Ligand-Mediated Cellular Activation

Aggregation of the intracellular domain of T cell zeta chain activates IL-2 production in T cells (Irving, B. A., and Weiss, A., “The cytoplasmic domain of the T cell receptor zeta chain is sufficient to couple to receptor-associated signal transduction pathways”, Cell, 64:891-901 (1991)). Cell lines may be established in which T cell signal transduction and IL-2 production (or the production of a product encoded by a reporter gene under NFAT transcriptional control) is stimulated by addition to the medium in which the cells are being maintained of a growth factor such as EPO. In such an engineered cell line, the ligand dimerizes its receptor, thereby aggregating the zeta subunit intracellular domain and activating NFAT-controlled transcription.

A receptor chimera is constructed by PCR or by site-specific deletion mutagenesis to encode the receptor ligand binding (extracellular domain) fused to the transmembrane and intracellular domain of the T cell receptor zeta chain. Methods for creating such a chimeric with the zeta chain are described in Irving and Weiss, Cell 64: 891 (1991) and Spencer et al. Science 262, 1019 (1993). Additional methods on creating cytokine receptor chimeras can be found in Fuh et al Science 256:1677 (1992). A cDNA encoding the receptor-zeta chain chimera is inserted into a mammalian expression vector, such as described by Spencer et al. The receptor expression vector is introduced into Jurkat cells, a T cell line, along with an IL-2 reporter gene under the control of the NFAT. Cells stably expressing both the receptor chimera and reporter gene are selected by G418 selection and detection of receptor on the cell surface by FACS.

Test compounds are incubated with the cells in the presence of the ligand. Compounds that bind to the receptor and interfere with ligand binding (and receptor activation) will block IL-2 production. Alternative reporters for NFAT-dependent gene expression can be used, such as beta-galactosidase, alkaline phosphatase or luciferase, in place of IL-2. Appropriate controls may be performed to eliminate molecules that act non-specifically. Coupling a receptor to the zeta chain in a stable cell line provides a much more sensitive functional assay than using primary cell bioassays. This also allows one to select a more robust cell type suitable for screening natural product and chemical libraries, Alternative cellular systems can be used such as the FDC-P1 cell line, dependent on G-CSF for growth (Fuh et al Science 256:1677 1992). Expression in FDC-P1 cells of a chimeric receptor containing a ligand-binding domain and the transmembrane and intracellular domain of the G-CSF receptor results in cells that are dependent on the ligand of interest for proliferation. Test compounds that interfere with the binding of ligand to receptor domain will block ligand-mediated cellular proliferation.

1. A method for preparing an agent capable of effecting a biological event mediated by the association of two or more endogenous protein mediator molecules which comprises covalently linking a first compound capable of binding to one of the protein mediators with a second compound capable of binding to the other protein mediator, to form a dimerizing agent capable of binding to both mediator molecules.

2. The method of claim 1 wherein the biological event is mediated by the association of two or more molecules of the same protein mediator and the first and second compounds are the same.

3. The method of claim 2 wherein the protein mediator is a cell surface receptor for a cytokine, growth factor or other hormone.

4. The method of claim 2 wherein the receptor is a receptor for EPO, G-CSF, TPO, GH, IL-2, interferon-alpha, interferon-beta, insulin or a neurotropic factor.

5. The method of claim 1 wherein the biological event is mediated by the association of molecules of two different protein mediators and the first and second compounds are different.

6. The method of claim 5 wherein the biological event is gene transcription, translocation of a selected protein to a predetermined cellular compartment, or destruction of a selected protein.

7. The method of any of claims 1-6 wherein the first and second compounds are non-peptidic.
